(19) United States
(12) Patent Application Publication
(10) Pub. No.: US 2013/0065823 A1
(43) Pub. Date: Mar. 14, 2013

(54) Supplement for Strengthening the Muscles of a Human

(71) Applicant: Sergio Ammirata, Coral Springs, FL (US)

(72) Inventor: Sergio Ammirata, Coral Springs, FL (US)

(21) Appl. No.: 13/232,032

(22) Filed: Sep. 14, 2011

(51) Int. Cl. A61K 38/17; A61P 21/00

(52) U.S. Cl. 514/5.7

(57) ABSTRACT

A protein and amino acid supplement for strengthening the muscles of a human, the supplement comprises of: a Creatine monohydrate; a vitamic C; a hydrolyzed collagen; and a hydrolyzed casein. The supplement may further comprise of L-glutamine, ribose, taurine, alpha lipoic acid, methyl sulfonyl methane. The supplement may further comprise of glucosamine sulfate and chondroitin sulfate.

### BACKGROUND

The present invention is directed to a protein and amino acid supplement used to strengthen the muscles of a human.

The protein and amino acid supplement of the present invention is the product of a treatment that the inventor conceived when treating his daughter for myopathy. His daughter suffered from a congenital myopathy.

Myopathy generally refers to any disease that affects human muscle tissue. Diseases of the muscle tissues result in weakness, inflammation, spasms, or paralysis. Myopathy can be the result of either inherited or acquired causes. The families of the infirmed are rendered helpless when it comes to the treatment of the diseases and the diseases are very painful to the infirmed.

From her birth until she was about 7 years old, his daughter was in and out of hospitals. Doctors performed countless tests on her, performed various medical procedures, and conducted various treatments to alleviate her pain. They managed to alleviate some of his daughter's pain, yet his daughter's condition did not appear to get better, for she continued to get weaker.

After watching the practitioners futile efforts to treat his daughter's myopathy, he decided to study what ailed his daughter on a more personal level. He hypothesized that his daughter suffered from a disease that did not allow his daughter's muscles to absorb proteins and/or amino acids. He hypothesized that if he could deliver proteins and amino acids to his daughter's muscle fiber cells that her muscles would strengthen, for the proteins and amino acids would serve as a catalyst to strengthen and regenerate her muscle fiber cells.

At first, he would simply give his daughter supplements that had high dosages of Creatine, glucosamine, and chondroitin. After trial and error, he realized that his daughter's muscle fiber cells were not absorbing the supplements. This caused him to conceive that he had to deliver the above compounds to his daughter's muscle fiber cells using another compound that would allow the muscle fiber cells to absorb the compounds.

Through trial and error, he eventually realized that when he delivered the above compounds using a hydrolyzed protein that his daughter started showing improvement. Hydrolyzed protein turned up to be the catalyst that he needed to deliver the above compounds to his daughter's muscle fiber cells.

His daughter was very weak when he first started giving her the supplement of the present invention. His daughter had a hard time lifting light things, riding a bicycle, and she suffered frequent hip displacements.

After a couple of months of taking the supplement of the present invention, his daughter's physical condition improved. She started to lift light items and her hip displacements became less frequent—from two episodes a day to one episode a week. After six months of taking the supplement, his daughter's pain was substantially reduced, she started playing with her friends and siblings, and she even started to run for substantial periods of time. The inventor of the present supplement believes that the supplement has helped his daughter live a more normal life.

His daughter's improvement after taking the supplement was noteworthy. For this reason, he decided that the supplement and his experience had to be shared with the world, for he remembered the helplessness that his family felt while watching the medical doctors treat his daughter.

For the foregoing reasons, there is a need for a protein and amino acid supplement to help strengthen and regenerate muscle fiber cells. The supplement might be used to treat all myopathies.

### SUMMARY

The present invention is directed to a protein and amino acid supplement that will help strengthen and regenerate muscle fiber cells. The supplement can be used to treat all myopathies.

The protein and amino acid supplement for strengthening the muscles of a human of the present invention is comprised of: a Creatine monohydrate; a vitamic C; a hydrolyzed collagen; and a Hydrolyzed casein. The supplement may further comprise of L-glutamine, ribose, taurine, alpha lipoic acid and methyl sulfonyl methane. The supplement may further comprise of glucosamine sulfate and chondroitin sulfate.

Creatine is an amino acid produced endogenously in the liver from arginine and glycine, and it is also found in meat products. Creatine phosphate is synthesized from Creatine and ATP, and it is catalyzed by Creatine kinase (CK). Unlike ATP, which the body is unable to store, Creatine phosphate can be stored to a limited degree in tissues, allowing for a supply of the high-energy phosphate bond, which can be utilized when needed. The hydration of phosphocreatine to Creatine and ATP thereby allows the ATP to be utilized by the tissue. Creatine is found in highest concentrations in skeletal muscle and to a lesser degree in cardiac muscle, smooth muscle, brain, sperm, and kidney. Creatine has not been found to have side effects and is being widely used by athletes to enhance performance.

The inventor hypothesized that individuals suffering from mytopathies may have lower levels of intramuscular phosphocreatine in their muscle fiber cells when compared to healthy individuals. He believes that by supplementing the muscle fiber cells with Creatine he can increase phosphocreatine within the cells. As stated above, phosphocreatine is an essential compound that is needed within muscle fiber cells for the ADP to ATP reaction to occur.

Glucosamine and Chondroitin are essential compounds that the connective tissues of the body must use. The formation of the intestinal mucosa and the production of hyaluronic acid for joint (synovial) fluids in the human body require glucosamine. As such, glucosamine has been used to offset the ravages of arthritis or osteoarthritis. Chondroitin is commonly used quite successfully in conjunction with glucosamine to offset the pains and inflammatory damage of arthritis and joint-related pain.

Hydrolyzed protein is protein that has been hydrolyzed or broken down into its component amino acids. While there are many means of achieving this, two of the most common are prolonged boiling in a strong acid (acid-HVP) or strong base, or using an enzyme such as the pancreatic protease enzyme to stimulate the naturally-occurring hydrolytic process. Unlike other forms of protein, a hydrolyzed protein is enzymatically broken down for quicker, more complete absorption. It is known that proteins are the building blocks for muscle development and restoration. Proteins absorption into the muscle fiber cells of the body of the infirmed will be increased by administering the hydrolyzed protein.

Hydrolyzed collagen is usually made from type I collagen by an enzymatic hydrolysis process. It is also called collagen hydrolysate, collagen peptide, gelatine, gelatine hydrolysate and hydrolyzed gelatine. Hydrolyzed collagen is produced from collagen found in the bones, skin, and connective tissue of animals such as cattle, pigs, horses, and fish. The process of hydrolysis involves breaking down the molecular bonds between individual collagen strands using heat and either acid or alkali solutions. Hydrolyzed collagen is a highly digestible protein, and an oral ingestion has been reported as having beneficial effects on joint & bone health, on skin health and for weight management and sports performance, particularly resulting in increased levels of energy.

The supplement of the present invention is made to replenish the normal levels of proteins and amino acids normally present in a healthy individual. Individuals suffering from myopathies should take the supplement for a period of at least 1 month prior to seeing any results. The inventor first noticed results with regards to his child approximately 2 months after initially administering the supplement.

The supplement is not a cure to myopathies. It is a supplement that is made to improve the quality of life of the infirmed.

An object of the present invention is to provide a supplement that can be used by muscle fiber cells to improve strength and regeneration.

Another object of the present invention is to provide a supplement that will improve the quality of life that an infirmed suffering from a myopathy.

Yet another object of the present invention is to provide a supplement that can be used as an alternative treatment to myopathies.

### DESCRIPTION

A protein and amino acid supplement for strengthening the muscles of a human, the supplement comprises of: a Creatine monohydrate; a vitamim C; a hydrolyzed collagen; and a Hydrolyzed casein.

In a further embodiment of the present invention, the supplement further comprises of L-glutamine, ribose, taurine, alpha lipoic acid and methyl sulfonyl methane.

In a further embodiment of the present invention, the supplement further comprises of glucosamine sulfate and chondroitin sulfate.

The embodiments of the present invention's Formulas (RATIO OF INGREDIENTS) are as follows:

| | | With | With Components | Allowed |
|---|---|---|---|---|
| | Base | components | Variation 1 and | Formula |
| Ingredients | Formula | Variation 1 | Variation 2 | Variarions |
| 1000 mg of Creatinine | 16.39% | 14.59% | 13.14% | 5% to 38% |
| Monohydrate | | | | |
| 100 mg of Vitamin C | 1.64% | 1.46% | 1.31% | 1% to 2% |
| 5000 mg Hydrolyzed Collagen/ | 81.97% | 72.97% | 65.68% | 25% to 60% |
| Hydrolyzed Casein | | | | |
| Additional Components/ | | | | |
| Variation 1 | | | | |
| 333 mg of L-Glutamine | | 4.86% | 4.37% | 3% to 25% |
| 166 mg of Ribose | | 2.42% | 2.18% | 1% to 4% |
| 166 mg of Taurine | | 2.42% | 2.18% | 1% to 4% |
| 4.16 mg of Alpha Lipoic Acid | | 0.06% | 0.05% | 0.02% to 0.09% |
| 83 mg of Methyl Sulfonyl Methane | | 1.21% | 1.09% | 0.50% to 1.75% |
| Additional components/ | | | | |
| Variation 2 | | | | |
| 500 mg of Glucosamine Sulfate | | | 6.57% | 3% to 10% |
| 260 mg of Chondroitin Sulfate | | | 3.42% | 1% to 7% |

Percentage values are calculated as a ratio of total weight of the components.

The supplement can be delivered to the infirmed in capsule, gel, liquid, powder, or any other form of delivery known in the art.

The recommended daily dosage of the supplement is as follows: The total weight of the supplement daily dosage will be calculated so that the total amount of Creatine delivered per day follows the following rule: Ages 2 through 6, 1 gram of Creatine per day. For ages 6 through 15, 1 gram per 40 lb of child weight per day (1×weight/40). Ages 15 or older, 5 grams of Creatine per day.

The supplement of the present invention can be administered to any infirmed suffering from any of the following diseases: myopathies; muscle weakness; chronic fatigue syndrome; arthritis; fibromyalgia; or muscular dystrophy.

An advantage of the present invention is that it will provide a supplement that will be used to improve the strength and regeneration of muscle fiber cells.

Another advantage of the present invention is that it provides a supplement that improves the quality of life of an infirmed suffering from a myopathy.

Yet another advantage of the present invention is that it provides a supplement that is used as an alternative treatment to myopathies.

Although the present invention has been described in considerable detail in reference to preferred versions, other versions are possible. Therefore, the spirit and scope of the appended claims should not be limited to the description of the preferred versions contained herein.

What is claimed is:

1. A protein and amino acid supplement for strengthening the muscles of a human, the supplement comprises of:
a Creatine monohydrate weighing from about 5 percent to about 38 percent of the total weight of the supplement;
a vitamic C weighing from about 1 percent to about 2 percent of the total weight of the supplement; and
a hydrolyzed casein and hydrolyzed collagen combination weighing from about 25 percent to about 60 percent of the total weight of the supplement.

2. The protein and amino acid supplement for strengthening the muscles of a human of claim 1, the supplement further comprises of:
an L-glutamine weighing from about 3 percent to about 25 percent of the total weight of the supplement;
a ribose weighing from about 1 percent to about 4 percent of the total weight of the supplement;
a taurine weighing from about 1 percent to about 4 percent of the total weight of the supplement;
an alpha lipoic acid weighing from about 0.02 percent to about 0.09 percent of the total weight of the supplement; and
a methyl sulfonyl methane weighing from about 0.50 percent to about 1.75 percent of the total weight of the supplement.

3. The protein and amino acid supplement for strengthening the muscles of a human of claim 2, the supplement further comprises of:
a glucosamine sulfate weighing from about 3 percent to about 10 percent of the total weight of the supplement; and
a chondroitin sulfate weighing from about 1 percent to about 7 percent of the total weight of the supplement.

4. A protein and amino acid supplement for strengthening the muscles of a human, the supplement comprises of:
a Creatine monohydrate weighing 1000 mg;
a vitamic C weighing 100 mg; and
a hydrolyzed collagen and hydrolyzed casein combination weighing 5000 mg.

5. The protein and amino acid supplement for strengthening the muscles of a human of claim 4, the supplement further comprises of:
an L-glutamine weighing 333 mg;
a ribose weighing 166 mg;
a taurine weighing 166 mg;
an alpha lipoic acid weighing 4.16 mg; and
a methyl sulfonyl methane weighing 83 mg.

6. The protein and amino acid supplement for strengthening the muscles of a human of claim 5, the supplement further comprises of:
a glucosamine sulfate weighing 500 mg; and
a chondroitin sulfate weighing 260 mg.

7. A protein and amino acid supplement for strengthening the muscles of a human, the supplement comprises of:
a Creatine monohydrate weighing about 16.39 percent of the total weight of the supplement;
a vitamic C weighing about 1.64 percent of the total weight of the supplement; and
a hydrolyzed collagen and hydrolyzed casein combination weighing about 81.97 percent of the total weight of the supplement.

8. A protein and amino acid supplement for strengthening the muscles of a human, the supplement further comprises of:
a Creatine monohydrate weighing about 14.59 percent of the total weight of the supplement;
an L-glutamine weighing about 4.86 percent of the total weight of the supplement;
a ribose weighing about 2.42 percent of the total weight of the supplement;
a taurine weighing about 2.42 percent of the total weight of the supplement;
an alpha lipoic acid weighing about 0.06 percent of the total weight of the supplement;
a vitamic C weighing about 1.46 percent of the total weight of the supplement;
a methyl sulfonyl methane weighing about 1.21 percent of the total weight of the supplement; and
a hydrolyzed collagen and hydrolyzed casein combination weighing about 72.97 percent of the total weight of the supplement.

9. A protein and amino acid supplement for strengthening the muscles of a human, the supplement comprises of:
a Creatine monohydrate weighing about 13.14 percent of the total weight of the supplement;
an L-glutamine weighing about 4.37 percent of the total weight of the supplement;
a ribose weighing about 2.18 percent of the total weight of the supplement;
a taurine weighing about 2.18 percent of the total weight of the supplement;
an alpha lipoic acid weighing about 0.05 percent of the total weight of the supplement;
a vitamic C weighing about 1.31 percent of the total weight of the supplement;
a methyl sulfonyl methane weighing about 1.09 percent of the total weight of the supplement;
a hydrolyzed collagen and hydrolyzed casein combination weighing about 43.78 percent of the total weight of the supplement;
a glucosamine sulfate weighing about 6.57 percent of the total weight of the supplement; and
a chondroitin sulfate weighing about 3.42 percent of the total weight of the supplement.
